(19) United States
(12) Patent Application Publication
(10) Pub. No.: US 2022/0125369 A1
(43) Pub. Date: Apr. 28, 2022

(54) DEVICES AND METHODS FOR MEASURING BRAIN STATE

(71) Applicants: Baylor College of Medicine, Houston, TX (US); Wisconsin Alumni Research Foundation, Madison, WI (US)

(72) Inventors: Matthew McGinley, Houston, TX (US); Justin Williams, Cambridge, WI (US); Aaron Suminski, McFarland, WI (US)

(73) Assignees: Baylor College of Medicine, Houston, TX (US); Wisconsin Alumni Research Foundation, Madison, WI (US)

(21) Appl. No.: 17/427,180

(22) PCT Filed: Feb. 26, 2020
(86) PCT No.: PCT/US20/19876

§ 371 Date: Jul. 30, 2021

Related U.S. Application Data
(60) Provisional application No. 62/813,978, filed on Mar. 5, 2019.

(51) Int. Cl. A61B 5/00; A61B 5/389; A61B 5/296

(52) U.S. Cl. CPC A61B 5/4064; A61B 5/389; A61B 5/0002; A61B 5/296; A61B 5/6821

(57) ABSTRACT

One aspect of the invention provides a method of measuring brain state. The method includes: receiving a first plurality of electrical signals from a contact lens placed on a surface of a subject's eye, the first plurality of electrical signals associated with a first plurality of electrodes lying adjacent to an iris dilator muscle of the subject's eye. Another aspect of the invention provides a contact lens including: an optically transparent or translucent substrate; and one or more pairs of electromyography electrodes arranged on or within the optically transparent or translucent substrate. At least one of the one or more pairs electromyography electrodes are arranged to lie adjacent to an iris dilator muscle of a subject's eye when the contact lens is placed on the eye.

### CROSS-REFERENCE TO RELATED APPLICATION

This application claims the benefit of priority to U.S. Provisional Patent Application Ser. No. 62/813,978, filed Mar. 5, 2019. The entire content of this application is hereby incorporated by reference herein.

### STATEMENT REGARDING FEDERALLY SPONSORED RESEARCH OR DEVELOPMENT

This invention was made with government support under Contract No. N66001-17-2-4010 awarded by the Defense Advanced Research Projects Agency. The government has certain rights in the invention.

### BACKGROUND OF THE INVENTION

Pupillometry (pupil diameter) can be used as a sensitive readout of brain state (sympathetic and parasympathetic) and its impact on perception and decision-making. Pupil diameter is controlled by the iris dilator muscle and sphincter muscle. Parasympathetic activity or light cause contraction of the sphincter muscles and pupil constriction.

### SUMMARY OF THE INVENTION

One aspect of the invention provides a method of measuring brain state. The method includes: receiving a first plurality of electrical signals from a contact lens placed on a surface of a subject's eye, the first plurality of electrical signals associated with a first plurality of electrodes lying adjacent to an iris dilator muscle of the subject's eye.

This aspect of the invention can have a variety of embodiments. The method can further include correlating the first plurality of electrical signals with a brain state.

The method can further include receiving a second plurality of electrical signals from the contact lens. The second plurality of electrical signals are associated with a second plurality of electrodes lying adjacent to an iris sphincter muscle of the subject's eye.

The method can further include comparing the first plurality of electrical signals with the second plurality of electrical signals. The method can further include normalizing the first plurality of electrical signals relative to the second plurality of electrical signals. The first plurality of electrical signals can be received wirelessly. The first plurality of electrodes can include pairs of diametrically opposed electrodes.

The method can further include receiving a third plurality of electrical signals from the contact lens. The third plurality of electrical signals are associated with a retina of the subject's eye.

Another aspect of the invention provides a contact lens including: an optically transparent or translucent substrate; and one or more pairs of electromyography electrodes arranged on or within the optically transparent or translucent substrate. At least one of the one or more pairs electromyography electrodes are arranged to lie adjacent to an iris dilator muscle of a subject's eye when the contact lens is placed on the eye.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1A depicts an eye.

FIG. 1B depicts an eye with electrodes overlaid according to an embodiment of the invention.

FIG. 2 provides schematics of exemplary electrode-bearing contact lenses according to embodiments of the invention.

FIG. 3 provides photographs of exemplary electrode-bearing contact lenses according to embodiments of the invention.

FIGS. 4-6 depict exemplary arrangements of electrode pairs on a contact lens according to embodiments of the invention.

FIG. 7 depicts a method of measuring brain state according to an embodiment of the invention.

FIG. 8 depicts an EMG measurement and recording system according to an embodiment of the invention.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

Embodiments of the invention provide contact lenses and methods of measuring brain activity.

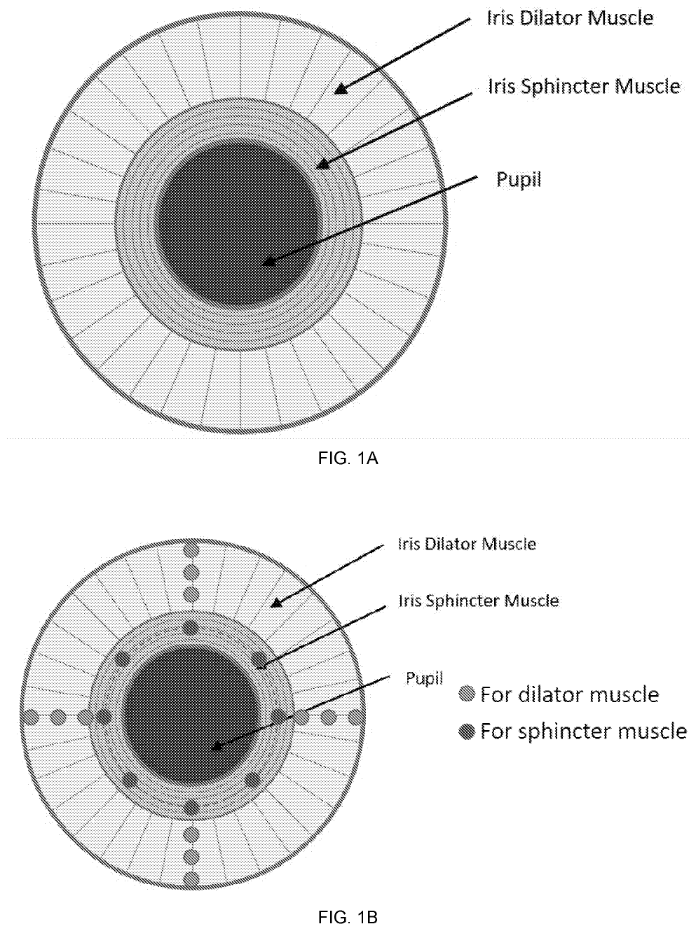

One embodiment of the invention provides a device to measure electromyography (EMG) via contact lens to gather information about the wearer's brain state. An EMG contact lens measures the muscle activity of the constrictor and dilator muscles in the pupil. Referring to FIG. 1A, the constrictor muscle provides insight into parasympathetic nervous system activity and is affected by light conditions. The dilator muscle provides information about the sympathetic nervous system, and is not affected by light conditions, thereby providing pure brain state information.

One application of this technology is the improvement of implantable brain devices, such as vagus nerve stimulators (VNS) and deep brain stimulators, by pairing the EMG lens with the implantable device to create a closed-loop system capable of responding to brain state changes with therapeutic intervention automatically. A potential application to space science or military is to identify and combat drowsiness and distracted attention. Other applications include brain state observations during sleep studies and commercial uses, such as interpreting brain state information in response to advertisements and marketing.

Compared to pupillometry as implemented with a camera, embodiments of the invention have several advantages including: (1) compatibility with natural environments, (2) robustness against ambient light, (3) ability to separate sympathetic and parasympathetic contributions, (4) potential to integrate measures of eye health, such as optic disc swelling, and (5) potential for practical clinical applications related to brain state, such as stress, circadian dysfunction, anxiety, depression, and other neurological or neuropsychiatric disorders.

### Exemplary Contact Lenses

Embodiments of the invention provide contact lenses including electrodes. The contact lenses can be corrective or non-corrective. The contact lenses can be rigid (e.g., glass, polymethyl methacrylate (PMMA), and the like), soft (e.g., silicone hydrogel), or hybrid (e.g., a rigid center surrounded by a soft “skirt”). The electrodes can be formed from a conductive material such as metals (e.g., gold, silver, titanium, stainless steel).

Applicants believe that electrode arrays as described in U.S. Pat. Nos. 8,386,007 and 9,861,288 and International Publication No. WO 2014/176240 can be adapted into contact lens form.

The contact lens can include a plurality of electrodes that extend to or through the lens material, one or more leads that convey electrical signals from the electrodes, and a processor coupled to the one or more leads. The processor can process the electrical signals on the contact lens or can receive and transmit the electrical signals. The processor can transmit signals through a wired or wireless (e.g., BLUETOOTH®, near-field communication) interface.

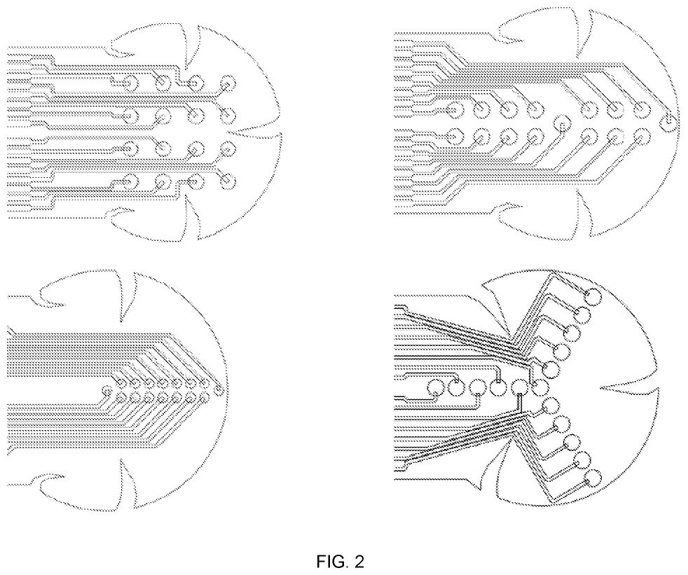

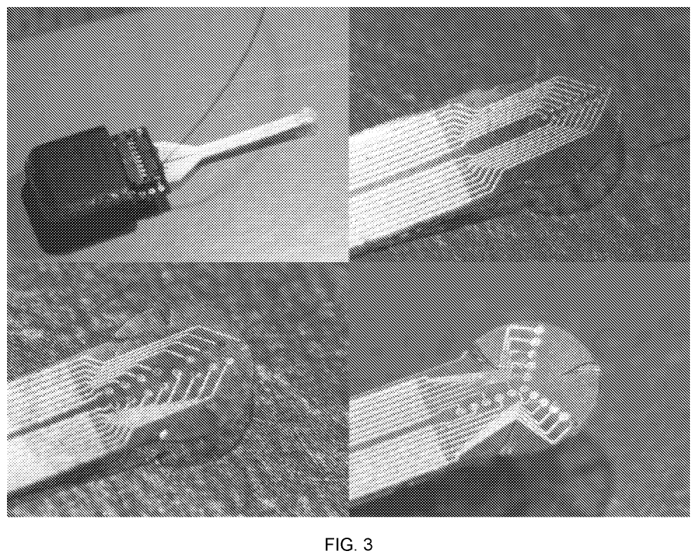

Exemplary electrode-bearing contact lenses are depicted in FIGS. 2 and 3.

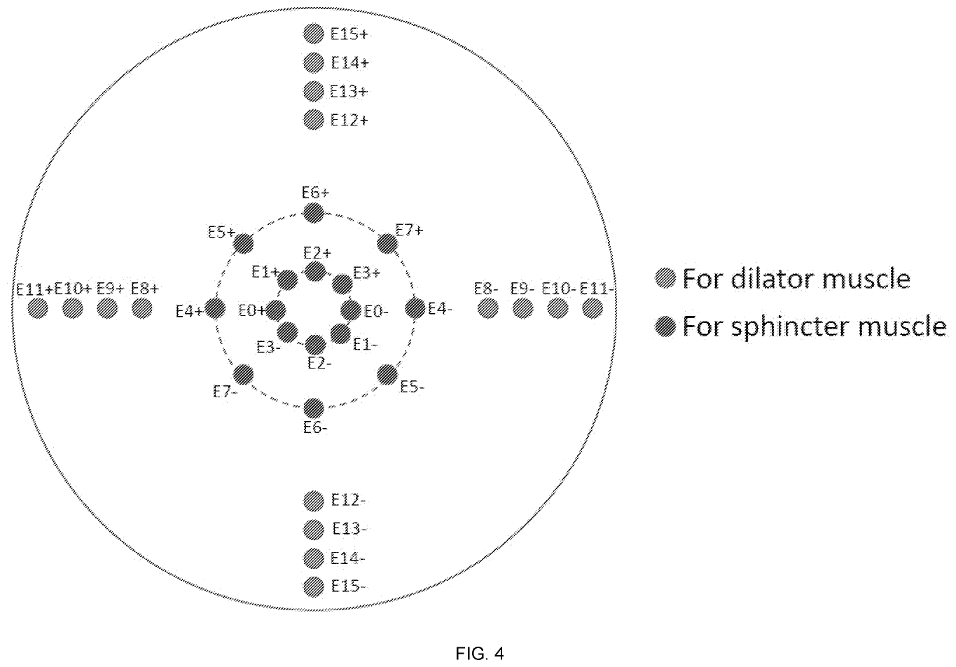

Referring now to FIG. 4, electrode-bearing contact lenses can include pairs of electrodes positioned to measure dilator and/or sphincter muscle activity. FIG. 4 depicts exemplary positions to measure each muscle, with electrode pairs E1-E7 positioned for measurement of sphincter muscle activity and dilator pairs E8-E15 positioned for measurement of sphincter muscle activity.

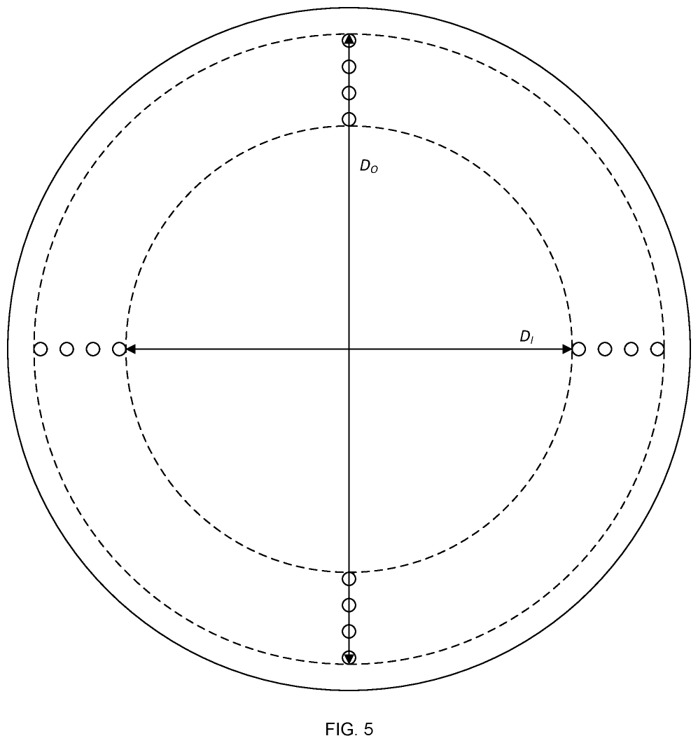

Referring to FIG. 5, another embodiment of the invention provides an electrode-bearing contact lens including electrodes focused exclusively on measuring dilator muscle activity. Such electrodes can be positioned within two concentric circles having diameters D_{O} and D_{I}. Although diameters may vary based on patient anatomy, an adult human pupil generally has a diameter of between about 2 mm and about 4 mm in bright light between about 4 mm and about 8 mm in dark light. Mouse pupils range from slight more than 0 mm in dark light to about 2 mm in dark light.

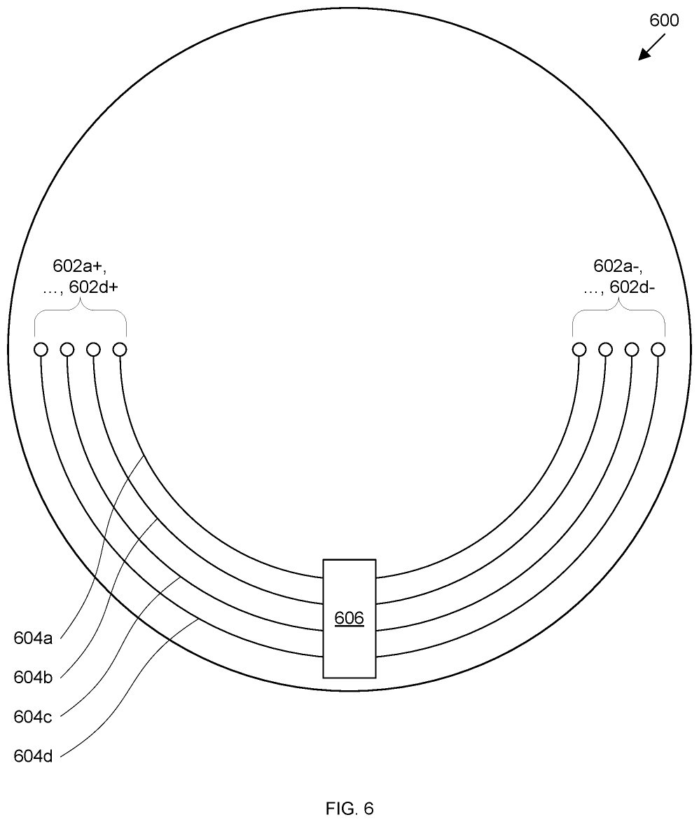

Referring to FIG. 6, an embodiment of the invention including four pairs of diametrically opposed electrodes 602 coupled to a transmitter 606 by leads 604. Advantageously, the electrode-bearing contact lens 600 does not include any opaque elements over the center of the contact lens, thereby avoiding any potential obstructions to vision.

The electrodes used to measure EMG can also be utilized to measure electroretinography (ERG), which can be used separately, in parallel with, or in combination with the EMG measurements described herein.

Embodiments of the invention can include a variety of other additional sensors within a contact lens such as electroretinogram or other electro-magnetic sensors, glucose sensors, photometers, imagers (e.g., of optic disk health), and the like.

### Exemplary Methods of Measuring Brain State

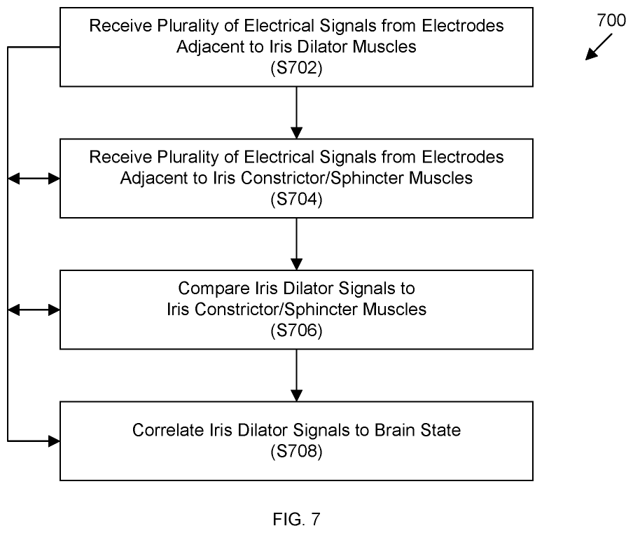

Referring now to FIG. 7, another embodiment of the invention provides methods of measuring brain state.

In step S702, a first plurality of electrical signals are received from a contact lens, such as the contact lenses described herein. The first plurality of electrical signals can be associated with a first plurality of electrodes lying adjacent to an iris dilator muscle of the subject's eye.

In step S704, a second plurality of electrical signals are optionally received from a contact lens, such as the contact lenses described herein. The second plurality of electrical signals can be associated with a second plurality of electrodes lying adjacent to an iris constrictor muscle of the subject's eye.

In step S706, the first plurality of electrical signals are optionally compared to the second plurality of electrical signals. Such comparison can assess whether the signals associated with dilator muscle are stronger than the signals associated with the sphincter muscles (e.g., in absolute terms or by a defined magnitude or factor). For example, the dilator muscle signals can be normalized based on the sphincter muscle signals.

In step S708, the first plurality of electrical signals and/or the second plurality of electrical signals can be correlated with brain state. Such correlations can be made based on previously measured iris EMG measurements and brain measurements such as through implanted electrodes, near-infrared spectroscopy (NIRS), functional near-infrared spectroscopy (fNIRS), magnetic resonance imaging (MM), functional resonance imaging (fMRI), and the like.

### WORKING EXAMPLE(S)

### Working Example 1—Hardware Implementation

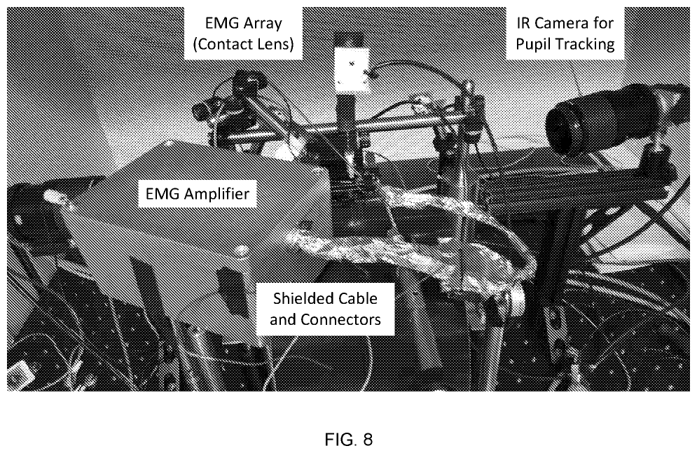

Referring now to FIG. 8, the EMG signal is measured and recorded with a commercial amplifier board and a USB interface board. The amplifier board has 16 bipolar channels for paired differential EMG recording. The USB interface board controls the amplifier board and streams EMG data to a host computer for storage and post analysis. A shielded custom cable and connectors were built for channel selection and for reducing measurement noise. Custom LABVIEW® software was completed for pupil data collection and for synchronization with the EMG data acquisition. The system has been fully integrated and is currently being used for experiments.

### PROPHETIC EXAMPLE(S)

### Prophetic Example 1—EMG Experiments

Applicants will continue the EMG experiments in mice. EMG measurements in one eye will be compared to pupil diameter measurements in the other eye, including pharmacological manipulations to isolate EMG signals from “contaminations” from extra-ocular muscles, retinal activity, and eye lid or face muscles. Applicants will then conduct experiments with larger species, including rats and rabbits. The bigger eyes from these species will allow Applicants to scale up the number of electrodes that can be used and most likely obtain EMG signals with higher amplitude and signal-to-noise ratio than in mice.

### Prophetic Example 2—Experimental Human Contact Lenses

Once the device development with the animal models is completed, Applicants will transition to humans. In this phase, materials and manufacturing process of the contact lens will be evaluated for biocompatibility and safety (e.g., corneal lesions, oxygenation, etc.). The contact lens will still be wired for EMG signal transmission. Experiment and data analysis techniques for shielding and artifact rejection will be developed to isolate the useful EMG signal.

### Prophetic Example 3—Commercial System Development

As the third development phase, integrated circuits (IC) will be designed and integrated with the contact lens. These circuits (e.g., including or coupled to an antenna) will be used for the EMG signal amplification and wireless transmission of the data to an external instrument. With such development, the hard-wired connection of the contact lens with the measurement system can be removed. This phase will bring the design closer to practical use in the field for consumer use.

Such devices can include a power source such as rechargeable battery, an inductive element, a photovoltaic cell, and the like. Such devices can also communicate with a computing device such as a smartphone, tablet, a watch and/or an activity tracker (e.g., devices sold under the APPLE WATCH® trademark by Apple, Inc. of Cupertino, Calif., the FITBIT® trademark by Fitbit, Inc. of San Francisco, Calif., and the like). Such integration can be facilitated by an app downloaded to the computing device.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A method of measuring brain state, the method comprising:
receiving a first plurality of electrical signals from a contact lens placed on a surface of a subject's eye, the first plurality of electrical signals associated with a first plurality of electrodes lying adjacent to an iris dilator muscle of the subject's eye.

2. The of method claim 1, further comprising:
correlating the first plurality of electrical signals with a brain state.

3. The method of claim 1, further comprising:
receiving a second plurality of electrical signals from the contact lens, the second plurality of electrical signals associated with a second plurality of electrodes lying adjacent to an iris sphincter muscle of the subject's eye.

4. The method of claim 1, further comprising:
comparing the first plurality of electrical signals with the second plurality of electrical signals.

5. The method of claim 1, further comprising:
normalizing the first plurality of electrical signals relative to the second plurality of electrical signals.

6. The method of claim 1, wherein the first plurality of electrical signals are received wirelessly.

7. The method of claim 1, wherein the first plurality of electrodes comprise pairs of diametrically opposed electrodes.

8. The method of claim 1, further comprising:
receiving a third plurality of electrical signals from the contact lens, the third plurality of electrical signals associated with a retina of the subject's eye.

9. A contact lens comprising:
an optically transparent or translucent substrate; and
one or more pairs of electromyography electrodes arranged on or within the optically transparent or translucent substrate, at least one of the one or more pairs electromyography electrodes arranged to lie adjacent to an iris dilator muscle of a subject's eye when the contact lens is placed on the eye.
